(19) United States
(12) Patent Application Publication
(10) Pub. No.: US 2004/0077986 A1
(43) Pub. Date: Apr. 22, 2004

(54) Human blood collection system

(76) Inventor: Alex F. Farris, Hoover, AL (US)

Correspondence Address: Robert J. Veal, 121 LaBlanc Way, Atlanta, GA, 30327, US

(21) Appl. No.: 10/426,677

(22) Filed: Apr. 30, 2003

Related U.S. Application Data
(60) Provisional application No. 60/354,625, filed on May 1, 2002.

(51) Int. Cl. G06F 17/60; A61M 5/00; A61B 19/00

(52) U.S. Cl. 604/7; 604/403; 705/2

(57) ABSTRACT

A method for collecting blood from identifiable groups wherein, the steps include: identifying a potential donor pool having common characteristics, identifying a specific stimulus to induce individual donors in the pool to donate blood, presenting the stimulus to the donors, creating a database including the names and addresses of the donors responding to the stimulus, matching the responding donors with geographically favorable collection centers, identifying active donors who donate blood at the collection centers, reinforcing the stimulus to the active donors, and periodically reminding the responding donors including the active donors to donate blood.

This application claims priority from U.S. patent application No. 60/354,625 filed May 1, 2002.

### FIELD OF THE INVENTION

The present invention relates to the field of health sciences and more particularly to the collection of blood from donors. Still more particularly the present invention relates to the stimulation of donors to give blood and to the rewarding and tracking of donors who give blood for subsequent donations.

### BACKGROUND OF THE INVENTION

Increasing population demands and the advances of medical science have lead to an ever-increasing demand for human blood for use in hospital surgical wards, emergency rooms and research facilities. Unfortunately, supply has not been sufficient to keep up with demand; therefore numerous attempts to emulate human blood with a synthetic blood have been advanced. Within the field of blood utilization, it is known to tag and identify blood by donor and to separate blood components for shipping. However, obtaining a supply of blood for usage remains a problem.

### SUMMARY OF THE INVENTION

The present invention has as its main object the provision of a safe and adequate supply of blood for the medical needs of the population.

Another object of the invention is the tracking and facilitating of donor response to blood donation requests.

Another object of the invention is the funding of higher education and the provision of scholarships to scholars based on the criteria selected by blood donors.

These and other objects of the invention will become apparent from a review of the description of the preferred embodiment, and a realization that a donor grouping based on related interests will provide a blood supply that can be harvested with regularity if that related interest group is given an incentive to supply blood, if the group is frequently stimulated to give, if the group is rewarded for giving, and if the group individuals can be identified for subsequent giving.

### DESCRIPTION OF THE DRAWINGS

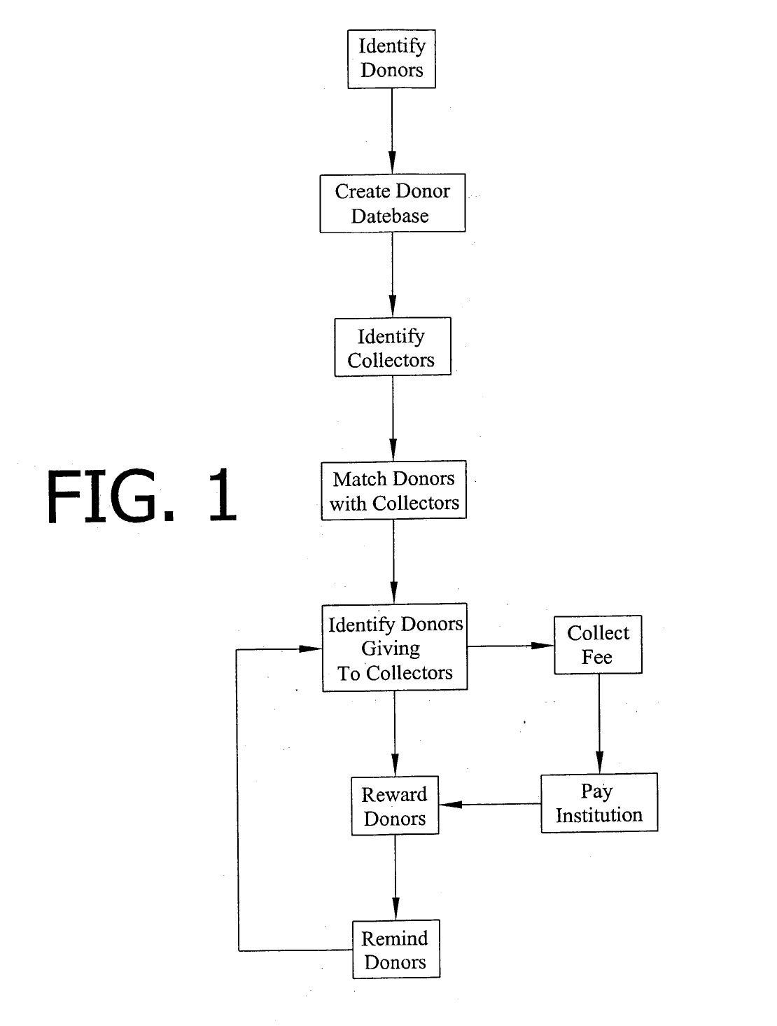

The method and apparatus used in the present invention is illustrated in the accompanying drawings, which form a portion of this disclosure and wherein FIG. 1 is a flow chart of the process.

### DESCRIPTION OF THE PREFERRED EMBODIMENT

Identification of donor pools allows the individual donor information to be input to a potential donor database, resident in an electronic monitoring system. Electronic monitoring system includes a network linked computer having at least memory, a programmable processing unit, and a programmable interface permitting access to said memory. Interest groups are first selected. Typical interest groups include: family members, alumni of boosters of academic institutions, veterans of military service, members of fraternal and professional organizations, members of service organizations, employees of organizations, sports fans, and the like. Routinely such interest groups have established communication channels such as newsletters, magazines, electronic chat rooms or bulletin boards, seminars and the like. Potential donors from the interest group may be identified by responses to stimuli presented through the normal communication channels such as electronic communication from the electronic monitoring system to the communication channels or through mass media, mailing list or the like.

The stimuli required for each group may vary, however, it is believed that the stimuli will be most effective when it is broadly based among the interest group. One stimulus is the funding of activities or projects of the interest group in combination with the overall worthiness of donating blood. To enable funding, the present invention would rely on commercial blood harvesting organizations that currently purchase blood for resale to hospitals and the like. One project is the provision of scholarships or other benefits. In the preferred embodiment, an interest group organization having an established communication channel is provided with a response eliciting communication in the form of a printed response card or an electronic posting a to a bulletin board from the monitoring system or from a system maintained by the organization, that advises the interest group of: 1. the urgent need for blood; 2. the activity or project of the group in need of funding; and, 3. the correlation between donating blood and the funding of the activity and project.

By way of example, a university alumni magazine or newsletter may include an insert card to be sent to a coordination entity. The response eliciting communication requires the donor to identify himself by name, address, age, affiliation with an interest group and or activity to which he wishes his donation to be credited. The coordinating entity utilizes the submitted information to create a potential donor database in the memory of the monitoring system. The coordination entity also creates a collection entity database including the names and addresses of each blood collecting entity participating in the program in memory of monitoring system, as well as an affiliated entity database. Cross referencing the addresses of the potential donors and the collection entities allows the coordination entity to generate a potential donor action communication which is sent to each potential donor advising the donor of the nearest collection agencies and their hours of operation.

Each collection agency is provided with limited access to the donor database in the monitoring system either by way of a coded electronic listing to be loaded into a software module on a computer at the collection agency or preferably through a distributed computer network connection managed by the monitoring system, the listing providing a listing of all current potential donors in a secure format such as an accessible lookup database that allows matching a donor with characteristics such as name, telephone number, or organizational identification number, a machine readable code sent to the donor from the coordinating entity. The database listing also includes the group affiliation of each potential donor and the address of the affiliated entity, which in this example is the affiliated university.

Each collection entity must have electronic means of communication between the monitoring system and a local computer such as a direct modem or terminal connection, an email connection, or other computer data exchange capability. A collection entity soft ware packet may provided for each collection entity, which is a specialized accounting and reporting packet for integration into the collection entities accounting system, including a data portion which is updated electronically at selected intervals. The accounting portion of the software is designed to tally the total number of units of blood donated on behalf of an affiliated group and by each individual, whereas the reporting packet is to iteratively identify responding donors to the affiliated group and coordinating entity and to report the information maintained in the accounting packet to the affiliated group and the coordinating entity and to any third party to the transaction.

Alternatively the interactive communication from the collection entity to the monitoring system database allows the monitoring system to perform all tracking based on data transmitted from the collection entity to the database. In this embodiment, the monitoring system can manipulate the database to determine the number of donors giving at each collection entity in the system and the affiliated organization or entity to whom the donor wishes to provide a benefit.

The collection agency must also transmit funds for each responding donor to the coordinating entity or the affiliated entity, which the collection agency recovers by the transfer of the collected blood to the hospital or medical facility. If the transfer of fluids from the collecting entity is through the coordinating entity then the coordinating entity is responsible for generating payment to the affiliated entity from the funds provided from the collecting entity

In the present example, each potential donor would go to the collection agency and identify himself. The collection agency upon entering the identification into the computer would be able to verify the identity of the individual donor and his affiliation and donor information, by interaction with a responding donor database located at the monitoring system. Alternatively, each collection entity may create locally a responding donor database would be periodically transmitted to the coordinating entity to update a master responding donor database stored in the memory of monitoring system.

In either embodiment, after the donor blood is taken, the collection agency database, donor database, affiliated entity database and any coordinating accounting entity databases are updated with the date of donation and other information such as blood type, or other medical data. Thus, in a preferred embodiment the process would include sending a machine or human readable message directing each potential donor to the location of a favorable collection center, entering data at the collection center when the responding donor becomes an active donor, updating the donor databases for each active donor, automatically reinforcing the stimulus of the active donors, updating the database for each donation made.

The collection entity or coordinating would then send information to the university identifying the donors in accordance with the privacy guidance provided by the donors, and the total number of units given. The university takes the individual information and provides a reinforcing stimulus in the form of a communication to the donor, public recognition of the donor, or similar reinforcement. The collection entity or coordinating entity would also forward funding to the University to be used in the selected activity, such as scholarship pools, or building programs. The coordinating entity would update the responding donor database with the date of donation and monitor the date such that a subsequent response card can be sent to the donor advising him that it is time to supplement his donation by giving more blood.

In another example, a family group may wish to set up a scholarship for a newborn infant. With the birth announcement, family members may be provided with response cards which would be utilized as above, however, the collection entity would report the participation by the family donors to a coordinating entity or directly to a financial institution or university to set up a scholarship for the identified newborn. Should the newborn not attend college then the funds transferred to the financial institution or university would be utilized or distributed in accordance with instructions provided by the parents of the child.

As may be seen, each interest group may have individual variations, however, the essential elements of the invention apply to all, namely, identifying a potential donor pool having common characteristics, identifying a specific stimulus to induce individual donors in the pool to donate blood, presenting the stimulus to the donors, creating electronic databases including the names and addresses of the donors responding to the stimulus, matching the responding donors with geographically favorable collection entities, identifying active donors who donate blood at the collection centers, reinforcing the stimulus to the active donors, and periodically reminding the responding donors including the active donors to donate blood.

What we claim is:

1. A method for supplying donor blood comprising the steps of identifying a potential donor pool having common characteristics, identifying a specific stimulus to induce individual donors in the pool to donate blood, presenting the stimulus to the donors, creating an electronic donor database including the names and addresses of the donors responding to the stimulus, matching the responding donors with geographically favorable blood collection entities, identifying active donors within said donor database who donate blood at the collection centers, reinforcing the stimulus to the active donors, and periodically reminding the responding donors including the active donors to donate blood.

2. A method as defined in claim 1 wherein the database is electronically stored and said matching and reminding steps are performed with a computer.

3. A method as defined in claim 1 wherein the common donor characteristic is support for a particular academic institution.

4. A method as defined in claim 1 wherein the common donor characteristic is a societal relationship to a newborn infant.

5. A method as defined in claim 1 wherein the common donor characteristic is membership in an identifiable organization.

6. A method as defined in claim 1 wherein the common donor characteristic is service in the military.

7. A method as defined in claim 1 wherein the common donor characteristic is age.

8. A method as defined in claim 1 wherein the common donor characteristic is national origin.

9. A method as defined in claim 1 wherein the common factor is determined by selective characteristic analysis.

10. A method as defined in any of claims 1 through 9 wherein the stimulus comprises the creation of a scholarship at an academic institution based on the donation of blood.

11. A method as defined in claim 10 wherein the stimulus comprises the creation of a scholarship for an identified individual at an academic institution.

12. A method as defined in any of claims 10 wherein the stimulus comprises the creation of a scholarship for unidentified individuals based on an interest common to the potential donors.

13. A method as defined in claim 1 further comprising sending each responding donor a human readable message directing said donor to the location of a favorable collection center containing a machine readable identifier, reading the identifier at the collection center when the responding donor becomes an active donor, automatically reinforcing the stimulus of the active donors based on reading of the identifier, updating the donor database for each message read.

14. A method as defined in claim 13 wherein reinforcing said stimulus comprises sending a specified value to an affiliated entity for accumulation with value received on behalf of other donors and advising said active donor of the receipt of the value at the affiliated entity.

15. A method as defined in claim 14 were in said reinforcing step includes publicly acknowledging the donation of blood by the active donors.

16. A method as defined in claim 1 wherein said reminding step comprises generating a message for each active donor advising said donor of the last date of donation and scheduling a subsequent date of donation.

17. A method as defined in claim 16 wherein said message is delivered electronically.

18. A method as defined in claim 16 wherein said message is printed for mail delivery.

19. A method as defined in claim 1 wherein the stimulus is the funding of a specific project undertaken by the interest group.
